(19) United States
(12) Patent Application Publication
(10) Pub. No.: US 2026/0272533 A1
(43) Pub. Date: Sep. 17, 2026

(54) CRYOGENIC FLUID COOLING AND STORAGE DEVICE, RECUPERATION DEVICE, AND RELATED SYSTEMS AND METHODS

(71) Applicant: CORFIGO, INC., Montclair, NJ (US)

(72) Inventor: Jason Sperling, Upper Saddle River, NJ (US)

(21) Appl. No.: 19/163,931

(22) PCT Filed: Mar. 11, 2024
(86) PCT No.: PCT/US2024/019387

§ 371 Date: Sep. 10, 2025

Related U.S. Application Data
(60) Provisional application No. 63/489,463, filed on Mar. 10, 2023.

(51) Int. Cl. A61B 18/02; A61B 18/00

(52) U.S. Cl. CPC A61B 18/02; A61B 2018/00577; A61B 2018/0231

(57) ABSTRACT

Cryogenic fluid cooling systems and methods for ablation procedures, including systems having at least one cooling container having a cryogenic fluid cooling unit and an interface detachably coupleable with an ablation device, wherein the cooling container is configured to cool the cryogenic fluid into a liquid state and wherein the system is configured to urge the cryogenic fluid via vapor pressure accumulation from the cooling container into the ablation device when the ablation device is coupled to the interface.

### CROSS-REFERENCE TO RELATED APPLICATION(S)

This application claims priority to International PCT Application No. PCT/US2024/019387, filed on Mar. 11, 2024, which claims the benefit under 35 U.S.C. § 119(e) to U.S. Application 63/489,463, filed Mar. 10, 2023 and entitled “Cryogenic Fluid Cooling and Storage Device, Recuperation Device, and Related Systems and Methods,” which is hereby incorporated herein by reference in its entirety.

### FIELD

The various embodiments herein relate to cryoablation systems, including cryoablation equipment for use with extremely low temperature cryogenic fluids.

### BACKGROUND

The use of cryoablation to treat heart rhythm disorders and malignant tumors has been long-established. Most of these tools utilize cryogenic fluid (gas or liquid) such as argon, nitrous oxide or liquid nitrogen. One disadvantage of known ablation systems and commonly-used cryogenic energy sources is that they are ineffective for ablating certain target tissues. More specifically, they are ineffective for the ablation of thick tissues (such as left ventricle muscle) and tissue that is warmed by an intimately associated physiologic “warmer” (such as the blood pool flowing in the cardiac chambers).

Thus, for ablation of the most demanding tissues discussed above, the optimal cryogenic fluids are those having lower boiling points than nitrogen, such as neon, helium, and hydrogen. More specifically, the low boiling point cryogenic fluids (also referred to herein as “extremely low temperature” or “XLT” cryogenic fluids) are those fluids with a boiling point that is lower than about 77 Kelvin (“K”).

However, there are disadvantages to the use of the low boiling point cryogenic fluids for cryoablation procedures in a hospital environment. For example, the use of hydrogen is not appropriate for clinical procedures, because it is highly flammable/explosive. In addition, helium is a scarce, irreplaceable resource that is difficult to source and expensive, making it less desirable to use helium as a cryoablation fluid in systems that exhaust the helium to the atmosphere. Further, the low boiling point cryofluids are difficult to store in liquid form because they often boil into gas despite excellent vacuum insulation because of their low boiling points. In addition, these XLT cryofluids are so cold that they are both cumbersome and dangerous to transfer from one vessel to another in known cryogenic systems (in contrast to relatively warmer cryofluids like liquid nitrogen that can be safely poured from one vessel to another in open atmospheric conditions). For example, the mandatory and formal instructions for transferring liquid helium from one suitable vessel to another require many steps and at least two people. Hospital personnel who manage cryoablation systems are not experienced in handling such low boiling point cryogenic fluids and are not trained to mitigate risks associated with cryofluid handling and transfer. Another disadvantage is that if an XLT cryogenic fluid is exposed to atmospheric conditions, it is so cold that it will liquefy the air in its vicinity (mostly the nitrogen and oxygen in the air), which thereby results in the contamination of the cryofluid. In fact, in the case of liquid helium, components of the ambient air not only liquefy but also solidify and can thereby obstruct flow pathways of the cryoablation system during cryofluid transfer or utilization. In addition, the liquefication and/or solidification of the components can also result in separation of those components, and pure oxygen in either liquid or solid form can be very dangerous. Finally, another disadvantage of the cryogenic fluids in cryoablation systems is their tendency to boil off under storage. For example, the normal evaporation rate of liquid helium using the best available insulation (a cryogenic liquid helium dewar, for example) is about 1% per day.

Standard cryoablation equipment also has disadvantages when XLT cryogenic fluids are used. For example, standard cryogenic fluid storage dewars are designed to function at low pressures, but a cryoablation system using low boiling point cryogenic fluid can require working pressures that exceed those low pressures. In a specific example, the CMSH Portable Liquid Helium Dewar, which is commercially available from Cryofab, has a maximum acceptable working pressure of about 10 psi, which is too low for use with XLT cryogenic fluids. Further, standard compressors typically used to pressurize a fluid in an ablation device cannot be used with XLT cryogenic fluids, because they generate heat during operation that inevitably warms the cryofluid, reducing the advantage of the cryofluid's extremely low temperature. In addition, most existing cryoablation systems allow depletion of the cryofluid—exhausting the spent cryofluid to the ambient environment. While there are known helium recirculation systems available, these systems are quite large and cannot achieve the coldest temperatures that are desirable for XLT cryoablation.

Finally, standard XLT cryogenic fluid recovery systems have another disadvantage: a large footprint that requires a great deal of space in the hospital environment. That is, XLT cryofluids have large liquid-to-gas expansion ratios. As a result, the known recovery systems for low boiling point cryogenic fluids require the use of large recovery balloons (3,000 L and larger) that contain the spent cryofluid and recover and liquefy the cryofluid over relatively long periods of time. Such a recovery system would not be viable in a hospital scenario because the space requirements would be very challenging. Similarly, the size of the standard compressors that would be required to generate the desired flow rates for XLT cryogenic fluid would be so large, loud, and heat generating as to be unusable in the procedure room.

There is a need in the art for improved cryogenic fluid cooling and storage devices, recuperation devices, and related systems and methods.

### BRIEF SUMMARY

Discussed herein are various cryogenic fluid cooling systems and methods for cooling cryogenic fluid for use in ablation procedures.

In Example 1, a cryogenic fluid cooling system for ablation procedures comprises a mobile unit comprising a first cryogenic cooling container comprising a first inner chamber, a cryogenic fluid cooling unit disposed within the first inner chamber, a first elongate outlet tube extending into the first inner chamber, the first elongate outlet tube comprising an outlet opening disposed at or near the bottom of the first inner chamber, and a first inlet tube extending into the first inner chamber, the first inlet tube comprising an inlet opening disposed at or near the cryogenic fluid cooling unit. The cryogenic fluid cooling system further comprises an interface associated with the mobile unit and detachably coupleable to an ablation device, the interface comprising an interface interior, and a transfer port in fluidic communication with the first inner chamber via the first elongate outlet tube, wherein the transfer port is removably coupleable with the ablation device. The first cooling container is configured to cool the cryogenic fluid disposed in the first inner chamber and retain the cryogenic fluid in a liquid or solid state for any period of time, and the system is configured to urge cryogenic fluid in the liquid state via vapor pressure accumulation from the first inner chamber into the ablation device via the transfer port when the ablation device is coupled to the interface.

Example 2 relates to the cryogenic fluid cooling system according to Example 1, wherein the vapor pressure accumulation occurs above a surface of the cryogenic fluid in the liquid state in the first inner chamber.

Example 3 relates to the cryogenic fluid cooling system according to Example 1, wherein the cryogenic fluid is exhausted in a gas state from the ablation device back into the cooling system.

Example 4 relates to the cryogenic fluid cooling system according to Example 1, wherein the cryogenic fluid is helium, neon, or hydrogen.

Example 5 relates to the cryogenic fluid cooling system according to Example 1, wherein first inner chamber comprises an inner wall configured to withstand internal pressures of up to around 100 psi.

Example 6 relates to the cryogenic fluid cooling system according to Example 1, further comprising a collection unit comprising a second cooling container comprising a second inner chamber and a second cryogenic fluid cooling unit disposed within the second inner chamber. The system further comprises an intake interface comprising at least one intake port coupleable with an exhaust cryogenic fluid source, wherein the at least one intake port is in fluidic communication with the second inner chamber, and a compressor unit operably coupled to the second cryogenic fluid cooling unit.

Example 7 relates to the cryogenic fluid cooling system according to Example 6, wherein the exhaust cryogenic fluid source is the ablation device or a container containing the exhaust cryogenic fluid from the ablation device.

Example 8 relates to the cryogenic fluid cooling system according to Example 1, further comprising a controller associated with the mobile unit, wherein the controller is configured to control flow of the cryogenic fluid within the system.

Example 9 relates to the cryogenic fluid cooling system according to Example 1, wherein the cryogenic fluid cooling unit comprises a cryocooler cold head unit.

Example 10 relates to the cryogenic fluid cooling system according to Example 1, wherein the system is configured to minimize or eliminate external loss of the cryogenic fluid.

Example 11 relates to the cryogenic fluid cooling system according to Example 1, wherein the system is configured to receive the spent cryogenic fluid from the ablation device and transfer the spent cryogenic fluid into at least one exhaust fluid container configured to receive the spent cryogenic fluid via a fluid container exhaust interface.

In Example 12, a cryogenic fluid cooling system for ablation procedures comprises a mobile unit comprising a removable cryogenic storage container comprising an inner chamber configured to be refillable with cryogenic fluid and an elongate outlet tube extending into the inner chamber, the elongate outlet tube comprising an outlet opening disposed at or near the bottom of the inner chamber. The system further comprises an interface associated with the mobile unit and detachably couplable to an ablation device, the interface comprising an interface interior and a transfer port in fluidic communication with the inner chamber via the elongate outlet tube, wherein the transfer port is removably coupleable with the ablation device. The removable cryogenic storage container is configured to retain the cryogenic fluid in a liquid state for any period of time. Further the system is configured to urge the cryogenic fluid in the liquid state via vapor pressure accumulation in the inner chamber into the ablation device via the transfer port when the ablation device is coupled to the interface.

Example 13 relates to the cryogenic fluid cooling system according to Example 12, wherein the vapor pressure accumulation occurs above a surface of the cryogenic fluid in the liquid state in the inner chamber.

Example 14 relates to the cryogenic fluid cooling system according to Example 12, wherein the cryogenic fluid is exhausted in a gas state from the ablation device back into the cooling system.

Example 15 relates to the cryogenic fluid cooling system according to Example 12, wherein the cryogenic fluid is helium, neon, or hydrogen.

Example 16 relates to the cryogenic fluid cooling system according to Example 12, wherein inner chamber comprises an inner wall configured to withstand internal pressures of up to around 100 psi.

In Example 17, a method of cooling, storing, supplying and recovering cryogenic fluid comprises cooling cryogenic gas with a cold head unit in a cooling container of a cooling system to a temperature of less than 27 Kelvin such that the cryogenic gas is converted into cryogenic liquid or solid, retaining the cryogenic liquid or solid in the cooling container for any period of time, transferring the cryogenic liquid into a supply container prior to an ablation procedure, transferring the cryogenic liquid into an ablation device via vapor pressure accumulation in the supply container, wherein the cryogenic liquid is converted into cryogenic gas as it passes through the ablation device, and receiving the cryogenic gas in the cooling system from the ablation device, wherein any external loss of the cryogenic fluid is minimized or prevented.

Example 18 relates to the method according to Example 17, wherein the cryogenic fluid is helium, neon, or hydrogen.

Example 19 relates to the method according to Example 17, further comprising immediately cooling the cryogenic gas received in the cooling system from the ablation device.

Example 20 relates to the method according to Example 17, further comprising cooling the cryogenic gas received in the cooling system from the ablation device in a cryocooler for future reuse.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a schematic view of a cryoablation system, according to one embodiment.

FIG. 1B is a schematic view of a recovery/liquefication container and a working supply container, according to one embodiment.

FIG. 2 is a schematic view of a recovery/liquefication container, according to another embodiment.

FIG. 3A is a schematic view of a cryogenic cooling and storage system, according to a further embodiment.

FIG. 3B is a schematic view of the working/supply unit of the system of FIG. 3A, according to one embodiment.

FIG. 3C is a schematic view of the recovery/liquefication unit of the system of FIG. 3A, according to one embodiment.

FIG. 4 is a schematic view of a recovery/liquefication container to be incorporated into the cryoablation system of FIG. 1A without a working supply container, according to one alternative embodiment.

### DETAILED DESCRIPTION

The various implementations herein relate to various cryogenic fluid cooling, liquefication, and storage device embodiments, recuperation device embodiments, and cryoablation systems having a cryogenic fluid cooling and/or storage device and a supply device. Further, certain of the various embodiments provide for not only cooling and storing a cryogenic fluid, but also are self-contained devices/systems that eliminate the need for cryogenic fluid to be regularly sourced and delivered to hospitals or any other patient treatment facilities, eliminate the need for complex and risky transfer of XLT cryogenic fluid from one container into another for a cryogenic ablation procedure, and/or eliminate the need to repeatedly connect/disconnect a cryogenic fluid container to/from an ablation system. In addition, various implementations can eliminate concerns related to having sufficient cryogenic fluid on hand to carry out ablation procedures due to delays in sourcing the cryogenic fluid and/or related to cryogenic fluid boiling off during storage. Finally, certain implementations provide for mitigation of cryogenic fluid waste during the required thaw cycles between successive ablations during a single procedure. Various devices and methods described herein allow the safe use of potent XLT cryofluids for ablation while maximizing XLT cryofluid recuperation and minimizing depletion. The XLT cryofluids that can be used in the various system embodiments herein include, but are not limited to, neon, helium, and hydrogen.

For purposes of this application, the term “cryogenic fluid” is intended to mean any liquid or gas that is intended for use in cryogenic ablation procedures. The term is not an indication of the specific temperature or phase (liquid or gas) of said cryogenic fluid at any particular time. Similarly, “XLT cryogenic fluid” is intended to mean any liquid or gas with a boiling point that is lower than about 77 K, but is not an indication of the temperature or phase of such a fluid at any particular time.

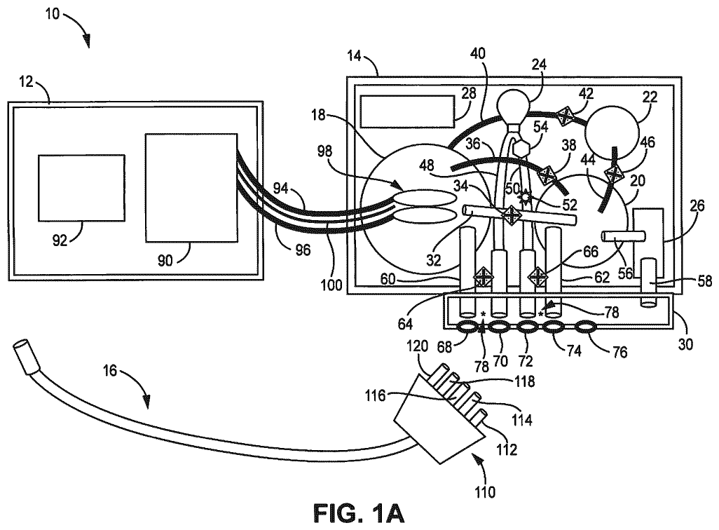

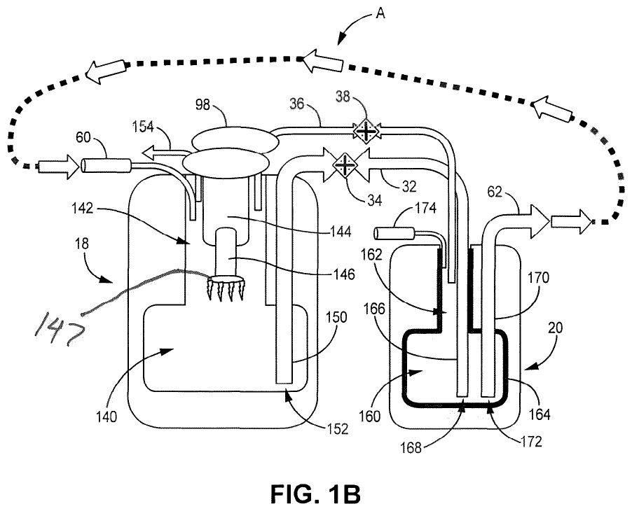

FIG. 1A shows an exemplary cryoablation system 10, according to one embodiment. The system 10 has a compressor unit 12, a container unit 14, and, in certain embodiments, a cryoablation device or system 16 that is coupleable to the container unit 14.

In some implementations, the container unit 14 has a cryofluid recovery/liquefication container 18, a working supply container 20, cryogenic fluid supply tank 22, a cryogenic gas accumulator 24, a vacuum unit 26, a controller 28, and an interface 30. The recovery/liquefication container 18 and the working supply container 20 are fluidically coupled via a first (or lance) connection line 32 having a valve 34 and a second (or boil-off) connection line 36 having a valve 38. The recovery/liquefication container 18 is fluidically coupled to the supply tank 22 via a supply connection line 40 having a valve 42, while the working supply container 20 is also fluidically coupled to the supply tank 22 via a working supply connection line 44 having a valve 46. The gas accumulator 24 has first and second warm connection lines 48, 50 extending therefrom, each of which extend to a respective port 70, 72 associated with the interface 30. The second warm line 50 has a warmer 52 and a compressor 54 associated therewith. A separate fluidic connection line (not shown) can also couple the second warm connection line 50 to the working supply container 20 such that the accumulator 24 is in fluidic communication with the working supply container 20 as well. The vacuum unit 26 is fluidically coupled with the working supply container 20 via a container vacuum line 56 and further is fluidically coupled with the interior of the interface 30 via an interface vacuum line 58. Further, the vacuum unit 26 is indirectly fluidically coupled with the recovery/liquefication container 18 via the second connection line 36 discussed above and the working supply container 20. In addition, a first cold connection line 60 is in fluidic communication with and extends from the recovery container 18 to a port 68 associated with the interface 30, and a second cold connection line 62 is in fluidic communication with and extends from the working container 20 to a port 74 associated with the interface 30. The first cold connection line 60 can be in fluidic communication with the first warm connection line 48 via a three-way valve or switch 64, while the second cold connection line 62 can be in fluidic communication with the second warm connection line 50 via a three-way valve or switch 66. The interface 30 also has a separate port 76 in fluidic communication with the interior of the interface 30. In addition, the container unit 14 can have temperature sensors 78 disposed adjacent to or within the lines 48, 50, 60, 62 to gauge the temperature of any fluid passing through those lines 48, 50, 60, 62. These temperature sensors 78 and all other sensors and valves in the system 10 can be coupled to the controller 28.

The controller 28 can be any known controller, including, for example, a computer 28. In certain implementations, the controller 28 has a graphic user interface (“GUI”) (not shown) that is coupled to and positioned on the controller 28 or positioned elsewhere on the container unit 14. Alternatively, the GUI (not shown) can be connected to the controller 28 and disposed remotely from the unit 14. A user can access and interact with the controller 28 via the GUI (not shown).

According to certain embodiments, the compressor unit 12 has a compressor 90 and a cooling unit 92. The cooling unit 92 can be used to cool the compressor 90 in a known fashion using water (via connecting water lines, which are not shown) urged between the cooling unit 92 and the compressor 90. The compressor 90 has fluid lines 94, 96 that extend from the compressor 90 to the cold head unit 98 associated with the recovery/liquefication container 18 such that the fluid lines 94, 96 are removably coupled with the cold head unit 98. As is common for operation of a cold head unit, the compressor 90 can be used to deliver helium to and from the cold head unit 98 via the fluid lines 94, 96. In addition, a cold head motor cord 100 extends between and is removably coupled to both the compressor 90 and the cold head unit 98 such that the compressor 90 can control the cold head motor (not shown) via the cord 100.

Further, in accordance with some implementations, the cryoablation device 16 can have a connection section 110 for coupling with the container unit 14. The connection section 110 has a vacuum line 112, a cold inflow line 114, a warm inflow line 116, a warm outflow line 118, and a cold outflow line 120. Hence, when the connection section 110 is coupled to the container unit 14 at the interface 30, the vacuum line 112 can fluidically couple with the interior of the interface 30 via the port 76, the cold inflow line 114 can fluidically couple with the second cold connection line 62 via the port 74, the warm outflow line 118 can fluidically couple with the second warm connection line 50 via the port 72, the warm inflow line 116 can fluidically couple with the first warm connection line 48 via the port 70, and the cold outflow line 120 can fluidically couple with the first cold connection line 60 via the port 68.

One specific exemplary embodiment of a recovery/liquefication container 18 and a working supply container 20 that can be incorporated into the container unit 14 is depicted in a side cross-sectional view in FIG. 1B. In this implementation, the recovery/liquefication container 18 has an inner chamber or reservoir 140 for collection of cryogenic fluid therein. The narrower top portion (or “neck”) 142 of the inner chamber 140 has the cold head unit 98 disposed therein. That is, the first stage 144 and the second stage 146 of the cold head 98 are both fluidically sealed within the neck 142. In one embodiment, the cold head unit 98, along with any cold head unit described elsewhere herein with respect to any embodiment herein, is any cryogenic fluid cooling unit for use in a recovery/liquefication container. One example of a container with a cold head unit that can be used for cold head unit 98 is a Cryomech Pulse Tube Cryocooler, which is commercially available from Bluefors (www.bluefors.com). Such a cold head unit 98 often has a known liquefier 147 associated therewith or incorporated therein. In accordance with various embodiments, the container 18 has a vacuum chamber or layer (not shown) disposed around inner chamber 140 and neck 142 and other standard insulation components or features in a fashion similar to standard cryogenic storage dewars. As discussed above, cold head 98 is removably coupleable to the compressor 90 such that the compressor 90 shuttles its own self-contained helium gas to the cold head 18 (via the fluid lines 94, 96) to cool the cold head 98, causing the stages 144, 146 to become extremely cold (about 4 K), thereby cooling any cryogenic fluid disposed within the inner chamber 140 that comes into contact with the stages 144, 146 and converting that fluid from a gas into a liquid.

The inner chamber 140 has an elongate tube (or “lance”) 150 disposed within the chamber 140 that extends toward the bottom of the chamber 140 such that the opening 152 of the lance 150 is disposed near the bottom of inner chamber 140. This position of the lance 150 ensures that the opening 152 is disposed well below the surface of any accumulated cryogenic liquid (which obviously accumulates at the bottom of the chamber 140 due to gravity) in the chamber 140. The lance 150 extends out of the container 18 such that it is in fluidic communication with the lance connection line 32, which is discussed above in relation to FIG. 1A. In addition, the boil-off connection line 36 (also discussed above in relation to FIG. 1A) extends into the inner chamber 140 such that the connection line 36 is in fluid communication with the inner chamber 140. Further, in certain embodiments, the container 18 can have at least one additional fluid line 154 that extends into the inner chamber 140 such that the line 154 is in fluidic communication with the inner chamber 140 and can be used to add cryogenic fluid to the inner chamber 140 or for any other purpose as discussed elsewhere herein. Shown also in FIG. 1B is the first cold connection line 60 (discussed above in relation to FIG. 1A), which extends into and is in fluidic communication with the inner chamber 140 as shown.

According to one embodiment as shown in FIG. 1B, the working supply container 20 also has an inner chamber or reservoir 160 for collection of cryogenic fluid therein. The narrower top portion (or “neck”) 162 of the inner chamber 160 can have certain fluidic lines extended therethrough as shown and as discussed in additional detail below. In accordance with various embodiments, the inner chamber 160 has a reinforced or relatively thick inner wall 164 that allows the inner chamber 160 to be subjected to working pressures that are substantially higher than typical working pressures in standard cryogenic dewars (of about 10 psi) without comprising the structural integrity of the wall 164. That is, according to one embodiment, the inner wall 164 is substantially thicker than the inner wall of the inner chamber 140 of the container 18. In certain implementations, the working container 20 has an inner wall 164 with sufficient thickness to allow the container 20 to operate in the about 50 to about 100 psi range. Further, the container 20 has a vacuum chamber or layer (not shown) disposed around inner chamber 160 and neck 162 and other standard insulation components or features in a fashion similar to standard cryogenic storage dewars.

The inner chamber 160 also has an elongate tube (or “lance”) 166 disposed within the chamber 160 that extends toward the bottom of the chamber 160 such that the opening 168 of the lance 166 is disposed near the bottom of inner chamber 160. As noted above with respect to the lance 150, this position of the lance 166 ensures that the opening 168 is disposed well below the surface of any accumulated liquid cryogenic fluid in the chamber 160. The lance 166 extends out of the container 20 such that it is in fluidic communication with the lance connection line 32, which is discussed above in relation to FIG. 1A, thereby resulting in the lance 166 being in fluidic communication with the lance 150 (with the valve 34 disposed in the connection line 32 as discussed above). In addition, the boil-off connection line 36 (also discussed above in relation to FIG. 1A) extends into the inner chamber 160 such that the connection line 36 is in fluid communication with the inner chamber 160, thereby resulting in the connection line 36 being in fluidic communication with both inner chambers 140, 160 (with the valve 38 disposed in the line 36). Further, the container 20 can optionally have at least one additional connection line 174 extending out of the container 20 that is in fluidic communication with the inner chamber 160 as shown.

The container 20 in this implementation also has a second elongate tube (or “lance”) 170 disposed within the chamber 160 that extends toward the bottom of the chamber 160 (like lance 166) such that the opening 172 of the lance 170 is disposed near the bottom of inner chamber 160. Like lance 166 and lance 150, this position of the lance 170 ensures that the opening 172 is disposed well below the surface of any accumulated liquid cryogenic fluid in the chamber 160. The lance 170 extends out of the container 20 such that it is in fluidic communication with the second cold connection line 62 (discussed above in relation to FIG. 1A). As such, when a cryoablation device (such as device 16) is coupled to the unit 14 as discussed above, cryogenic fluid can be urged out of the container 20 through the lance 170, the second cold connection line 62, and into the cold inflow line of the ablation device (such as line 114 of the ablation device 16 as discussed above). Thus, in addition to the two direct connection lines 32, 36 discussed above that provide direct fluidic communication between the two containers 18, 20, there is a third fluidic connection between the two containers 18, 20 when an ablation device is connected to the system. That is, as best shown with the arrows A in FIG. 1B, when an ablation device (such as device 16) is coupled to the system (such as system 10 as shown in FIG. 1A), a fluidic line or loop is established that extends from second cold connection line 62, through interface port 74 and cold inflow line 114, through the ablation device 16, and then out of the ablation device 16 via cold outflow line 120, interface port 68, first cold connection line 60, and into the inner chamber 140 of the container 18.

In certain alternative embodiments, the working supply container 20 can also have a standard cold head unit or other cryogenic fluid cooling unit (not shown) similar to that described above in relation to the recovery/liquefication container 18 that can operate in a similar fashion as described herein. In some implementations, a system containing such a working supply container 20 would have no need for the connection lines 32, 36 and the containers 18, 20 could have different internal temperatures based on the cold head units (such as unit 98) therein.

In operation, prior to coupling the system 10 to a cryoablation device (such as device 16), the system 10 can be “charged up,” which means that the appropriate amount of cryogenic fluid is cooled to the desired temperature. To do this, the cryogenic fluid (which can be gaseous or liquid helium in some cases) can be urged into the recovery/liquefication container 18 from an external source. For example, in one exemplary implementation, the cryogenic fluid is urged from a standard external high-pressure tank (cylinder or cylinders) to the container 18 via any one of the fluid lines that is in fluidic communication with the inner chamber (not shown) of the container 18. In this specific example, the external tank (not shown) can be coupled to the first cold connection line 60 via port 68 and the cryogenic fluid from the tank is slowly fed into the container 18. In one embodiment, the cryogenic fluid is room temperature gas in the tank and is urged into the container 18 under low pressure (less than about 5 psi, in certain embodiments).

While the cryogenic fluid is being urged into the container, the cold head 98 is actuated such that it gets extremely cold. In certain embodiments, the cold head 98 can reach about 4.2 K. In an exemplary alternative in which the cryogenic fluid is neon, the cold head 98 can reach a temperature of about 27 K or less. Thus, over time, the cold head 98 cools the cryogenic fluid in the container 18 to the point that it converts from gas into liquid. Once a sufficient volume of cryogenic liquid has been rendered, the cryogenic gas supply to the container 18 is stopped. In one embodiment, the sufficient amount of cryogenic liquid can be about 30 L or more. Alternatively, the desired amount of liquid can be any known amount as needed for a particular procedure.

During any time period that the system 10 is retained in storage/pre-procedure status, the system 10 can continue to maintain a desired amount of cryogenic liquid in the liquefication container 18. More specifically, as long as the system 10 is plugged into electrical power, it can be set to an “intermittent mode” or “maintenance mode.” This maintenance mode can be maintained for any period of time. In this mode or setting, the system 10 is designed to ensure that any internal cryogenic liquid boil-off (that is, evaporation of the cryogenic fluid) in the container 18 is automatically reliquefied. In one embodiment, this automatic reliquefication is accomplished using pressure sensors (not shown) disposed within the container 18 (or in a component in fluidic communication therewith) and coupled to the controller 28 or a separate controller associated with the compressor 90. That is, the pressure sensors (not shown) can be used to track the pressure within the container 18 such that when the pressure in the inner chamber 140 exceeds a predetermined level, the system 10 automatically actuates the cold head unit 98 to begin cooling the cryogenic fluid in the chamber 140 until enough gas is converted back into liquid to lower the pressure to a desired level (at which point the cold head unit 98 is shut off). In this fashion, all of the infused and converted cryogenic liquid is retained, and none is allowed to boil off and vent outside of the system 10.

In preparation for an ablation procedure, a volume of the cryogenic liquid in the liquefication container 18 must be transferred into the working/supply container 20. In contrast to known devices/procedures in which the cryogenic fluid must be manually transferred by two trained individuals (including manually inserting lances or stingers into each of the two containers), in the instant implementation, the recovery/liquification container 18 is already permanently connected to working/supply container 20 via permanently embedded lances 150, 166, as best shown in FIG. 1B. In certain implementations, the transfer of the cryogenic liquid to the working/supply container 20 can be controlled by the controller 28 (and—in some cases—by a user operating the controller via the GUI discussed above). Prior to the transfer, according to some implementations, the working/supply container 20 can be evacuated using the vacuum unit 26, which applies a vacuum via the container vacuum line 56. Once the container 20 is evacuated, the valve 34 in the lance connection line 32 is opened (either manually or by the controller 28), and the cryogenic liquid is urged from container 18 into container 20 either by the vacuum/pressure gradient and/or by allowing a pressure head of cryogenic fluid vapor to accumulate above the liquid level in the inner chamber 140 of the container 18. As a result, the cryogenic liquid passes from container 18 via embedded lance 150 into container 20 via embedded lance 166. In one implementation, the pressure head can accumulate by internal boil-off (before the pressure trigger enables re-liquification) or can be added exogenously to container 18 via the supply tank 22, connection line 40 and valve 42. Once the working/supply container 20 is filled with the desired and/or predetermined amount of cryogenic liquid, the valve 34 is closed. In certain embodiments, the container 20 is filled to its nominal capacity. Alternatively, any desired amount of cryogenic liquid can be transferred. At this point, the system 10 is ready to be used with a cryoablation device. Thus, the system 10 is disconnected from the electrical supply in its storage area (and any other connections) and transported into the procedure room.

Once in the procedure room, the system 10 is reconnected to electrical power and the cryoablation device 16 is coupled to the container unit 14. More specifically, the connector section 110 is coupled to the interface 30 such that the lines 112-120 are coupled to the ports 68-76 as discussed in detail above. In certain implementations, in the procedural arena, the compressor unit 12 can be positioned at some distance from the container unit 14 for user convenience. In such implementations, the fluid lines 94, 96 and motor cord 100 are sufficiently long to allow for such distance. In those embodiments in which the controller 28 is used to operate the system 10, the controller 28 can be set to a “procedure mode” at this point in the process such that instructions and interactions take place via the GUI.

Once the ablation device 16 is coupled to the unit 14, a vacuum is applied to the interior of the interface 30 such that the first cold connection line 60 and the second cold connection line 62 are insulated via the vacuum and minimize any heat leak. More specifically, the vacuum unit 26 is actuated to apply the vacuum via interface vacuum line 58. Further, the first cold connection line 60, the first and second warm connection lines 48, 50, and the second cold connection 62 are evacuated prior to the ablation procedure. It is necessary to evacuate all air from any lines that connect to the ablation device 16 in order to avoid air solidification in the lines and obstruction of cryofluid flow. In this specific embodiment, the vacuum unit 27 has vacuum connection lines (not shown) in communication with the valves 64, 66 coupled to the cold and warm connection lines 48-62. As such, the vacuum unit 27 can apply a vacuum to those lines 48-62 via those open valves 64, 66, thereby evacuating them. Alternatively, vacuum lines can be fluidically coupled to the lines 48-62 in any other fashion such that the vacuum unit 27 can be used to evacuate those lines. Once the lines 48-62 are evacuated, the valves 64, 66 are closed. The application of the vacuum to the interior of the interface 30 also results in the application of a vacuum to the annular space of the ablation device via the port 76 and vacuum line 112, thereby insulating the cryogenic fluid lines therein.

In this embodiment, during the ablation procedure, the cold head unit 18 is running continuously and on full power. Further, prior to starting an ablation procedure, a vacuum can be applied to the space in the inner chamber 140 of the container 18 above any residual liquid level. In one implementation, the vacuum is applied by the vacuum unit 26 via the valve 64 in communication with the first cold connection line 60. And when a threshold vacuum level is achieved in the inner chamber 140, the vacuum is turned off. At this point, the ablation procedure can begin.

When the ablation procedure begins, a working pressure must be established in the working/supply container 20. According to one embodiment, the working pressure can range from about 50 psi to about 100 psi (which is far less than known high-pressure cryogenic systems). In those implementations in which a user can use the controller 28, the user selects the appropriate settings for the cryoablation cycle to be initiated, including the establishment of the working pressure. In one embodiment, the working pressure is established by actuating the supply tank 22 to supply a small amount of cryogenic gas to the inner chamber 160 of the working/supply container 20 via working supply connection line 44 and the valve 46. Alternatively, the cryogenic fluid can be supplied to the container 20 from the accumulator 24 and compressor 54 (which can be fluidically coupled to the container 20, even though that is not shown in FIG. 1A). Regardless of the source, the cryogenic gas urged into the container 20 pushes down on the liquid surface in the inner chamber 160, which in turn urges cryogenic liquid to pass from the bottom of the inner chamber 160 into lance 170 that is in fluidic communication with second cold connection line 62, which is fluidically connected to the cold inflow line 114 of the ablation device 16 via the vacuum-insulated interface 30.

As the cryogenic liquid enters the ablation device 16 via the cold inflow line 114, the liquid boils into extremely cold vapor as it passes along the pathway to the tip of the device 16, after which the gas returns to the system 10 via the cold outflow line 120 of the device 16 and the first cold connection line 60. At this point, according to some embodiments, a limited volume of cryogenic gas returning from the ablation device 16 may be allowed to pass via the valve 64 and first warm connection line 48 into the accumulator 24 for later use. In certain implementations, this flow can be controlled by the controller 28, and when temperature sensors 78 detect a predetermined temperature threshold (in which the temperature has fallen to such predetermined level), the valve 64 is actuated to divert very cold exhaust gas into recovery container 18 via first cold connection line 60. As the cold exhaust gas passes over or past the running cold head unit 98, the gas is quickly cooled such that it is colder, denser, and takes up less volume.

This quick cooling of the exhaust gas allows the current system 10 (and all system embodiments disclosed or contemplated herein) to retain a certain volume of cryogenic fluid in comparison to known systems. That is, during a cold ablation cycle, between 1 to 2 L of cryogenic liquid is converted to extremely cold gas as it passes through the ablation device (such as device 16). In the exemplary system 10, the cryogenic fluid is exhausted back into the recovery/liquefication container 18, and the cold head unit 98 (maintaining a temperature of about 4 K) quickly cools that cryogenic fluid to a very cold temperature such that the cryogenic fluid contracts to a volume that can be retained within the inner chamber 140. In other words, the current system embodiments disclosed or contemplated herein have a unique cooling container that makes it possible to transfer 1 to 2 L of cryogenic liquid into an ablation device and transfer the gas exhaust back into the recovery/liquefication container 18 and cool that so quickly that an amount of cryogenic fluid can be recovered in a much smaller container than is possible with known systems. In contrast, in known systems, the collection location of the cold exhaust gas is warmer than the exhaust gas such that the gas will continue to warm and expand and cannot be contained in anything but a very large container.

Thus, in further detail and in contrast to the known systems, in this exemplary embodiment, the exhausted cold gas remains as cold as possible as a result of the vacuum insulation in the interface 30, and the cold-head unit 98 in the recovery container 18 maintains a temperature of about 4 K. As a result, the incoming very cold exhaust, which may be between about 10 and 25 K, is actively cooled upon entry into recovery container 18. Because, in certain exemplary embodiments, each cold ablation cycle can take about 4 minutes and ramps up to highest helium mass flows over that interval, the cold head 98 has some time to work on the incoming gas. And as noted above, this allows the system 10 to handle more cryogenic fluid than known systems. Because the cold head unit 98 is maintained at about 4 K, equilibrium will ultimately be reached and the helium ‘mass’ will become colder and contract because its density will increase. Further, after each cold cycle of the ablation device is completed, there is a “rest period” during which no exhausted gas enters the recovery container 18 for several minutes. During this time, the cold head 98 can work even more efficiently at cooling the contents in the inner chamber 140. Thus, before the next cold cycle, the collected gas in the chamber 140 is much colder and denser compared with when it initially entered the container 18. Thus, depending on the size of the inner chamber 140 and the cooling power of the cold head 98, some amount, or even all of the cryogenic liquid originally supplied by supply container 20 can be recovered. Finally, once all of the cold cycles of the ablation procedure have been completed, the recovery/liquification container 18 can “finish the job” and liquefy any residual collected/recovered cryogenic fluid over several hours after the procedure.

According to certain embodiments, between each cold cycle during an ablation procedure, a warm/thaw cycle is provided so that the tip of the ablation device (such as device 16) can be removed from the last target (to overcome the “cryoadhesion” that occurs when the ablation tip adheres firmly to the target tissue). In certain prior art ablation devices/systems, instead of a warm cycle, resistive warming elements are included in the tip of the ablation element to address the cryoadhesion, but they add bulk and can be either slow or difficult to control or even add unnecessary risk. In contrast, the various system embodiments herein allow for delivering a warm cycle after each cold cycle. More specifically, the system 10 utilizes the same cryogenic fluid delivery fluid lines in the ablation device 16 to deliver warm helium gas to the ablative element of the ablation device 16. In accordance with certain implementations, the controller 28 can be used to cycle between the extremely cold cryogenic fluid and warming fluid by actuating the valves 64, 66. More specifically, when it is time for a warm/thaw cycle, the valve 66 is actuated to cut off the flow of cold cryogenic fluid through the second cold connection line 62 and further to open the flow of warm fluid via the second warm connection line 50. At the same time, the valve 64 is actuated to close off cold exhaust via the first cold connection line 60 and further to open the flow of warm exhaust fluid via the first warm connection line 48. Once the valves 64, 66 have been actuated as described above to open the flow of warm fluid, the compressor 54 urges fluid flow through the second warm connection line 50 such that the warming fluid passes through warmer 52. From there, the warm gas is delivered from the second warm connection line 50, through port 72, and into the warm inflow line 116 in the connector 110 of the ablation device 16. The warm gas passes to the tip of the ablative element of ablation device 16 and then back through warm outflow line 118, through port 70, and into first warm connection line 48. In one embodiment, the warm gas is warmed to about 37° C. Alternatively, any known warming temperature can be used. This flow of warm fluid continues until the temperature sensors 78 in interface 30 indicate that the warm exhaust fluid temperature is warm enough to indicate that the ablative element of the ablation device 16 can be safely detached from the target tissue. According to one embodiment, the target temperature of the warm exhaust fluid is about 0° C., but it could be other known temperatures as well. At this point, the ablation device (such as device 16) can be moved and re-positioned using standard navigation techniques in order to find the next tissue ablation target. Once it is positioned as desired, another cold cycle is initiated.

After each cold cycle and warm cycle, the valve 46 can be closed such that flow from the supply tank 22 is cut off, thereby resulting in the absence of both cold flow and warm flow.

When the ablation procedure is complete, the ablation device 16 is uncoupled from the container unit 14. More specifically, the connector 110 is removed from interface 30. This leaves the ports 68-76 exposed. In certain embodiments, a removable storage interface plug (not shown) is coupled to the ports 68-76 such that the ports 68-76 and associated lines are covered and fluidically sealed off from the ambient air. Further, in some implementations, the plug can create a fluidically sealed connection between the second warm connection line 50 and the first cold connection line 60.

At this point, the system 10 may be moved back into its storage location, where it is re-connected to an electrical supply. In those embodiments in which the controller 28 is used to operate the system 10, the controller 28 can be used to place the system 10 in “storage/recuperation mode.” Upon placement back in storage, the compressor 90 continues to operate at full power until the cold head unit 98 liquefies all of the cryogenic gas stored in the recovery container 18. Once the system 10 detects—via the pressure sensors discussed above—that all of the gas within the inner chamber 140 has been liquefied, the compressor 54 is actuated to drive gas stored in accumulator 24 under low pressure (less than about 5 psi in certain embodiments) into recovery container 18 via the storage interface plug (not shown) discussed above and connection lines 50, 60. After all this stored cryogenic fluid is recuperated/liquefied, the first cold connection line 60 is cut off via valve 64 and the boil-off connection line 36 is opened via its valve 38 so that any boil-off of the remaining cryogenic liquid contained in supply/working container 20 can be recovered and liquefied in the recovery container 18. At this point, the system 10 can be switched to intermittent mode as described above to re-liquefy boil-off in both containers 18, 20. The system 10 remains in this mode or status until the next ablation procedure approaches, at which point the cryogenic fluid is once again transfer to the supply/working container 20 as described above and the process begins again.

As such, the various exemplary embodiments disclosed or contemplated, including not only the embodiment of FIGS. 1A and 1B, but also the subsequent embodiments described below, relate to systems capable of substantial recovery of all utilized cryogenic fluid in a self-contained, automated system that is portable. In other words, the various system implementations herein minimize or eliminate any venting or loss of cryogenic fluid from the system. According to certain embodiments, the systems herein may need small amounts of cryogenic fluid to be replenished from time to time from an exogenous gas supply.

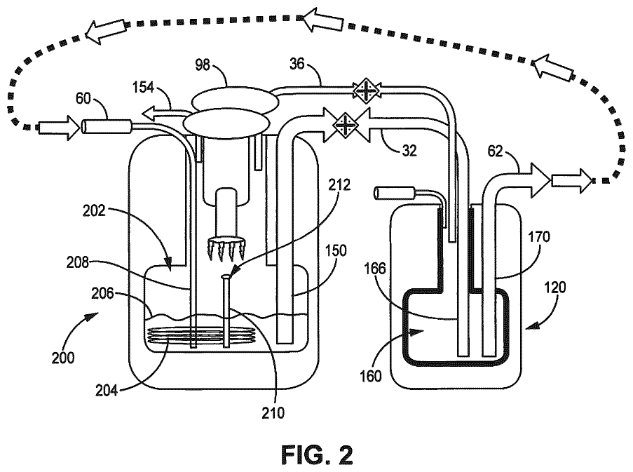

An alternative implementation of a recovery/liquefication container 200 is depicted in FIG. 2. Except as expressly discussed below, the various components displayed in FIG. 2 and/or discussed herein are substantially similar—having the substantially the same structure and function—as the components depicted in FIGS. 1A and 1B and discussed above (and have the same reference numbers). In this embodiment, the container 200 has a heat exchanger 204 disposed in the bottom of the inner chamber 202 such that the heat exchanger 204 can be submerged in any cryogenic liquid 206 or solid that is present in the chamber 202 as shown. In one embodiment, the heat exchanger 204 is a length of metal (such as copper, for example) tubing that is formed into several loops within the chamber 202. Alternatively, the heat exchanger 204 can be any known heat exchanger 204 for use in such cooling systems. Further, the container 200 has an intake tube 208 that extends from the first cold connection line 60 into the inner chamber 202 of the container 200 such that the tube 208 extends towards the bottom of the chamber 202 and is coupled directly to (or integral with) the heat exchanger 204 that is underneath a volume of liquid or solid cryogen 206 such that the inner lumen of the intake tube 208 is in direct fluidic communication with the inner lumen of the heat exchanger 204. Further, a tube extension 210 is coupled to the heat exchanger 204 at an opposite end of the exchanger 204 in relation to the tube 208 and extends upward within the chamber 202 such that the opening 212 of the tube extension 210 is disposed at the end of the extension 210 and is in fluidic communication with the inner chamber 202. In operation, as the cold cryogenic fluid exhaust from the ablation device (such as device 16) enters the system 10, it passes through the first cold connection line 60, through the intake tube 208, through the heat exchanger 204, through the tube extension 210, and out of the opening 210 such that it enters the chamber 204. When the liquid or frozen cryogen 206 is colder than the exhausted gas entering tube 208, as it passes through the heat exchanger 204, the cryogenic exhaust gas is super-cooled by the heat exchanger 204. In certain embodiments, the exhaust gas is cooled to close to about the temperature of liquid helium, which is about 4.2 K. Alternatively, the exhaust gas can be cooled to any temperature up to and including 4.2 K. In other embodiments, instead of a heat exchanger 204, the exhausted gas is allowed to directly contact a surface area volume of exposed frozen cryogen 206 that is colder than the exhaust gas, thereby cooling it so that it can liquefy. In an exemplary embodiment using neon as the cryogenic fluid, liquid neon (around 25-27K) is urged from container 120 into the ablation device coupled at cold connection 62. When the neon has passed through the ablation device and returns to collection unit 200 as cold neon vapor, it would pass via tube 208 which would terminate above a volume of neon that has been frozen to around 4K. As the neon exhaust strikes the frozen neon 206, it may liquefy or solidify for recovery.

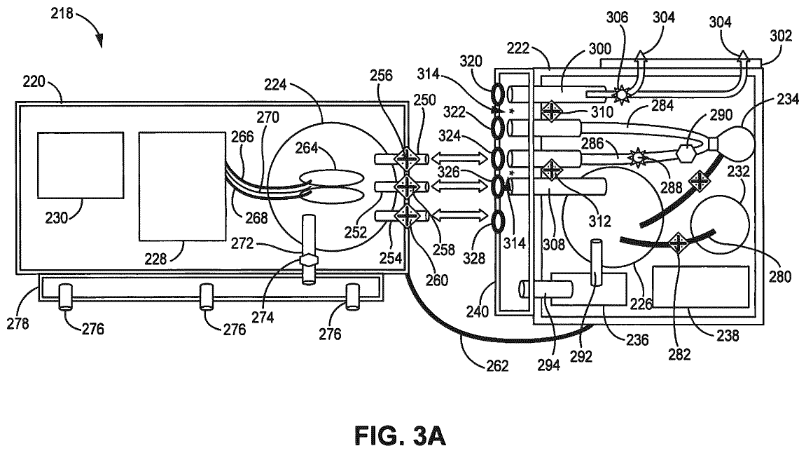

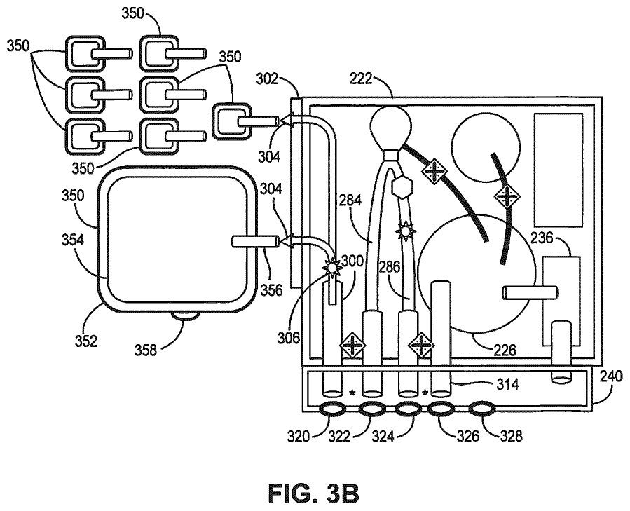

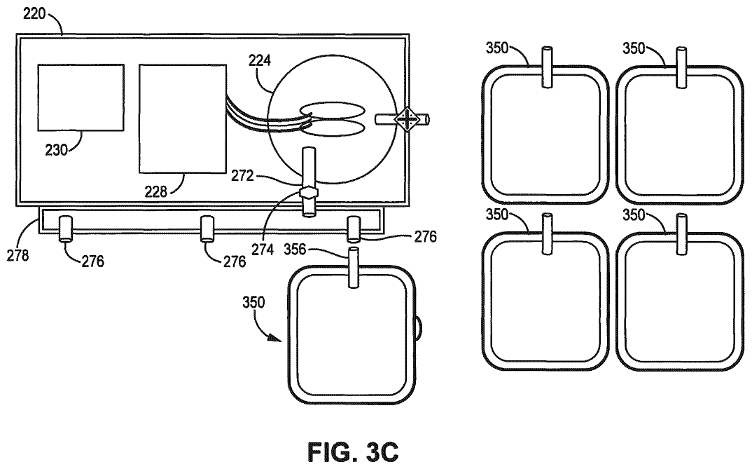

Another alternative implementation of a cryogenic cooling and storage system 218 is depicted in FIGS. 3A-3C. As with the other embodiments herein, this exemplary system 218 can be used to cool and store cryogenic fluid and operate in conjunction with an ablation device while minimizing or completely preventing any cryogenic fluid loss. Unlike the system 10, in this specific implementation, the system 218 is separated into a recovery/liquification unit 220 and a working/supply unit 222 such that the system 218 can help to save space and reduce system bulk/noise in a procedure room. Except as expressly stated herein, the various components of this system 218 are substantially similar to their corresponding components in system 10 and have the substantially the same components, features, and functionality.

The recovery/liquefication unit 220 contains the recovery/liquefication container 224, the compressor 228, and the cooling unit 230. The compressor 228 is coupled to the container 224 via fluid lines 94, 96 and a cold head motor cord 270 that extend from the compressor 228 to the cold head unit 264 associated with the recovery container 224. The unit 220 also has three fluid connection lines 250, 252, 254 in fluidic communication with the recovery container 224. The fluid connection line 250 is warm intake communication line 250, the fluid connection line 252 is the lance extension communication line 252, and the fluid communication line 254 is the vacuum communication line 254. Each of the lines 250, 252, 254 has a valve 256, 258, 260 associated therewith. In addition, there is also a cryogenic fluid intake line 272 with a compressor 274 associated therewith. The intake line 272 (and compressor 274) are in fluidic communication with a set of ports 276 on a manifold 278 that are configured to couple with exhausted cryogenic fluid containers, as will be discussed in detail below. In one embodiment, the ports 276 are valved (not shown) and are reversibly capped when not in use. In certain implementations, the recovery/liquefication unit 220 can also have a vacuum unit (not shown) that can be in fluidic communication with the recovery/liquefication container 224 to apply a vacuum thereto as desired when the unit 220 is not operably coupled to the working/supply unit 222.

The working/supply unit 222 contains the working/supply container 226, a cryogenic fluid supply tank 232, a cryogenic gas accumulator 234, a vacuum unit 236, a controller 238, and an interface 240. The working supply container 226 is fluidically coupled to the supply tank 232 via a working supply connection line 280 having a valve 282. The gas accumulator 234 has first and second warm connection lines 284, 286 extending therefrom, each of which extend to a respective port 322, 324 associated with the interface 240. The second warm line 286 has a warmer 288 and a compressor 290 associated therewith. The vacuum unit 236 is fluidically coupled with the working supply container 226 via a container vacuum line 292 and further is fluidically coupled with the interior of the interface 240 via an interface vacuum line 294. In addition, a first cold connection line 300 is in fluidic communication with a port 320 associated with the interface 240 and further extends to exhaust ports 304 in the recovery manifold 302. In some embodiments, the connection line 300 can have a warmer 306 associated therewith. Further, a second cold connection line 308 is in fluidic communication with and extends from the working container 226 to a port 326 associated with the interface 240. The first cold connection line 300 can be in fluidic communication with the first warm connection line 284 via a valve 310, while the second cold connection line 308 can be in fluidic communication with the second warm connection line 286 via a valve 312. The interface 240 also has a separate port 328 in fluidic communication with the interior of the interface 240. In addition, the working/supply unit 222 can have temperature sensors 314 disposed adjacent to or within the lines 300, 284, 286, 308 to gauge the temperature of any fluid passing through those lines 300, 284, 286, 308. These temperature sensors 314 and all other sensors and valves in the system 218 can be coupled to the controller 238.

These two units 220, 222 are reversibly attachable via the fluid connection lines 250, 252, 254 (discussed above) and the communication cable 262. During storage and in preparation for ablation, the units 220, 222 are connected, and a vacuum can be applied to the recovery dewar 224 via the vacuum communication line 254 by the vacuum unit 236 via the interface 240. In certain embodiments, the controller 238 can control this evacuation by actuating the vacuum unit 236 and opening the valve 260. Any interface components that are not used when the units 220, 222 are coupled together are fluidically sealed with an interface plug (not shown).

In use, the system 218 is stored and prepared for an ablation procedure in a fashion relatively similar to system 10 discussed above. In order to prepare the system 218 for an ablation procedure, cryogenic gas is supplied via any of the cryogenic gas ports 65 of the manifold 278 such that the gas enters the inner chamber of the container 224 via the cryogenic fluid intake line 272. In certain embodiments, the coupling of the cryogenic gas source to one of the ports 276 actuates the compressor 274, which helps to transfer the cryogenic gas into the container 224. When the cryogenic gas enters the container 224, it is liquefied via the cold head unit 264 as previously described. In accordance with some implementations, the initial loading with cryogenic gas and conversion to liquid for use in an ablation procedure can take up to several days in this system 218 (and in system 10 in certain embodiments), since a large volume of cryogenic liquid is generated. Subsequent recovery of the spent cryogenic gas after the procedure is faster because only a modest percentage of the total cryogenic liquid is used in any single procedure. Once a predetermined amount of cryogenic liquid is converted from gas within the container 224, the system 220 can be set to “intermittent mode” to re-liquefy its own internal boil-off as previously described.

Before a planned procedure, the cryogenic liquid is transferred into the working/supply container 226 in the working/supply unit 222. In certain embodiments, the controller 238 can control the transfer, because to the controller 238 can control elements of recovery unit 220 via the communication cable 262. Vapor pressure is allowed to accumulate above the cryogenic liquid level in the container 224, and/or a vacuum is applied to the container 226 by the vacuum unit 236 via vacuum line 292. At this point, the valve 258 can be opened and cryogenic liquid can flow by vacuum/pressure gradient from container 224 into container 226 via lance extension communication line 252. Once sufficient cryogenic fluid is transferred into the container 226, the system 218 is ready for the ablation procedure. At this point, the valves 256, 258, 260 can be closed, the units 220, 222 can be separated and unit 222 can be moved into a procedure room.

In this implementation, once the unit 222 has been moved into the procedure room, the unit 222 has a smaller footprint in comparison to the unit 14 of system 10 discussed above such that the working/supply unit 222 takes up less space in the room. Despite the smaller size, the unit 22 functions substantially in the same manner as the unit 14 of system 10 when connected to a cryoablation device, with the various steps described in detail above (with respect to system 10). However, in certain embodiments, when the ablation device (such as device 16 discussed above and depicted in FIG. 1A) is coupled to the interface 240, the cryogenic fluid can be urged from the working/supply container 226 into the ablation device via gas vapor pressure accumulation by delivering cryogenic gas from high-pressure gas container 232 into the neck portion of container 226 via connecting line that pushes the fluid through the ablation device.

Further, the handling of the cold cryogenic exhaust from the ablation device is handled differently with unit 222, as best shown according to one embodiment in FIG. 3B. More specifically, instead of the cold connection line 60 being in fluidic communication with the recovery/liquefication container 18 as in system 10, in the current system 218 embodiment, the first cold connection line 300 is in fluidic communication with the ports 304 such that any exhaust cryogenic gas can be directed out of the unit 222 whereby the gas can be collected in one or more external containers.

Exemplary external containers (or “collection units”) 350 are depicted in FIG. 3B. Such containers 350 may be of any shape, including cuboid or cylindrical. In one embodiment, each of the containers 350 has dimensions of about 28″×28″×60″, which holds a volume of around 770 L (roughly the expanded volume of a single liter of liquid helium). Alternatively, each of the containers 350 can have any known dimensions for such a container. The larger container 350 in the figure (which is depicted as larger to indicate that it contains cryogenic fluid therein) has an outer layer 352 and inner layer 354, for better protection from puncture or rupture. These collection units 350 can also have at least one valved recovery connection line 356 that traverses both layers 352, 354 and can fluidically couple to any of the ports 304 in the recovery manifold 302. Further, in certain alternatives, the units 350 can also have a pressure relief valve (not shown). As the ablation procedure progresses, the cold cryogenic gas exhaust is conducted from the ablation device into the first cold connection line 300 and may be warmed by warmer 306 as it passes thereby before exiting through one of the ports 304 into a collection unit 350. In certain embodiments, the warmer 306 warms the exhaust gas such that the gas is warm enough that it can be handled outside the unit 222. Alternatively, in other embodiments, collection units can be used that have insulation or other insulative components or features that allow for the units to contain colder exhaust gas while still being capable of being handled (and also can hold more helium because of the greater density of the colder helium and thus can be smaller as well). According to some implementations, the gas flow can be facilitated by an additional small compressor (not shown). The recovery manifold 302 can have two or more ports 304 so that at least two collection units 350 can be attached thereto at any given time, since it is expected that it will likely require between one and two collection units 350 to collect the spent cryofluid of a single cold cycle. For purposes of the full cryoablation procedure, in some embodiments, multiple empty collection units 350 are made available in the procedure room (as represented by the multiple units 350 in FIG. 3B). As each one is filled, the nursing/technical staff in the room can detach full collection units 350 from the ports 304 and move them to another part of the room or another room (such as, for example, the storage room where the cryocooler/liquefier unit 220 resides). For example, the full units 350 can be removed and replaced with empty units 350 during the warm/thaw cycles, which can take several minutes and allow sufficient time for such an exchange. Each of the collection units 350 can, in accordance with certain embodiments, have a handle 358 for ease of transport.

Each of the collection units 350 can then be coupled to the recovery/liquefication unit 220 in the storage room to transfer the spent cryogenic gas back into the recovery/liquefication container 224, as shown in FIG. 3C. That is, each of the multiple collection units 350 can be coupled to a port 276 along the manifold 278. As noted above, according to some implementations, the coupling of the connection line 356 of the container 350 to the port 276 can automatically trigger actuation of the compressor 274 so that the recovered gas can be transferred under low pressure via the intake line 272. In accordance with certain embodiments, each collection unit 350 can have a valve (not shown) in the connection line 356 that ensures that when the collection unit 350 is evacuated of its gas, it will remain evacuated and collapsed.

Once all the collection units 350 have been emptied, the working/supply unit 222 can be returned to the storage room and reconnected to the cryocooler/liquefier unit 220 via lines 250, 252, 254, 262. Once it's reconnected, the valves and compressors within the system 218 can be actuated (by the controller 238, for example) to direct any un-recovered helium gas from unit 222 (such as helium in accumulator 234, for example) into container 224 for liquification, as discussed above. The system 218 can then be set to “intermittent/storage” mode so that internal boil-off from both containers 224, 226 can be re-liquefied as also discussed above. As the next procedure approaches, the valves and other components will be actuated to facilitate transfer of cryogenic liquid from container 224 to container 226 yet again, and the process can be repeated as needed.

A further alternative implementation similar to the cryogenic cooling and storage system 10 of FIG. 1 or system 218 of FIGS. 3A-3C is also contemplated. Except as expressly stated herein, the various components of this alternative system are substantially similar to their corresponding components in system 10 or system 218 and have the substantially the same components, features, and functionality. In this alternative system, only a working/supply unit (similar to unit 222) is provided, and the working/supply container (similar to container 226 as discussed in detail above) is removable from the unit 222 and replaceable. Thus, in use, a separate (uncoupled) working/supply container (similar to 226) can be filled with cryogenic fluid—either on location or at another location—and then connected to the working/supply unit (similar to 222). Once the container is coupled to the system, the unit (similar to 222) can be positioned in the procedure room, coupled to the ablation device, and the ablation procedure can be performed in a fashion similar to that described above.

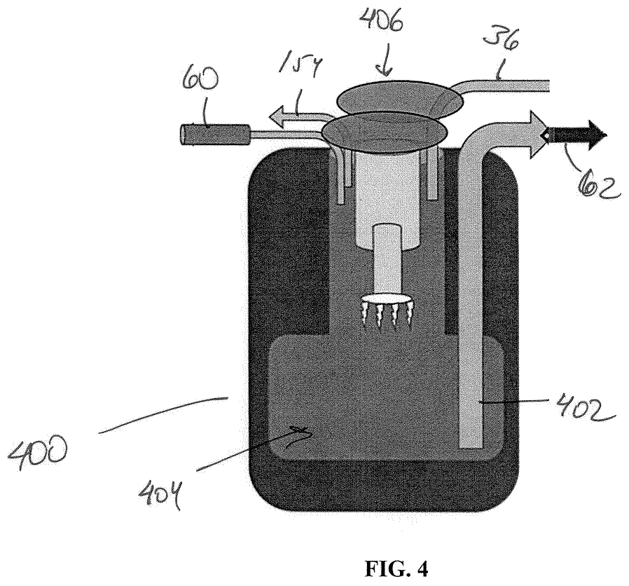

Returning to FIG. 1A, an alternative embodiment of the cryoablation system 10 is contemplated in which the container unit 14 has a working/supply container 400 with an embedded cold head unit 406 as shown in FIG. 4 (but has no recovery container). Except as expressly discussed below, the components and features of this alternative system implementation having no working supply container will be substantially similar to the components and features discussed above with respect to FIGS. 1A and 1B. With reference to FIG. 1A in combination with FIG. 4, the working/supply container 400 of this implementation is fluidically coupled to the supply tank 22 via a supply connection line 40 having a valve 42. While not shown in the figure, the working/supply container 400 is also fluidically coupled directly to second cold connection line 62 in this embodiment. In this embodiment, the fluid lines 94, 96 of the compressor 90 and the motor cord 100 extend from the compressor 90 to the cold head unit 406 associated with the working/supply container 400.

Turning now to FIG. 4, the elongate tube (or “lance”) 402 extends out of the container 400 such that it is in fluidic communication with the second cold connection line 62, as mentioned above. As such, when a cryoablation device (such as device 16) is coupled to the unit 14 as discussed above, cryogenic fluid can be urged out of the container 400 through the lance 402, the second cold connection line 62, and into the cold inflow line of the ablation device (such as line 114 of the ablation device 16 as discussed above). While not shown, the metal lining of inner chamber 404 will be thicker than usual, as in FIG. 1B (64), so as to accommodate vapor pressure accumulation in inner chamber 404 that can urge the cryofluid out via lance 402. Thus, when an ablation device (such as device 16 is coupled to the system, a fluidic line or loop is established that extends from the inner chamber 404 through the lance 402, through the second cold connection line 62, through interface port 74 and cold inflow line 114, through the ablation device 16, and then out of the ablation device 16 via cold outflow line 120. Cold exhaust may be either recovered by any method previously described, or collected by the system to be warmed and exhausted elsewhere (not shown).

In operation, prior to coupling the system 10 to a cryoablation device (such as device 16), the system 10 can be “charged up” in a fashion described above such that cryogenic fluid is urged into the container 400 and cooled to the point that it converts from gas to liquid. At this point, the system 10 is ready to be used with a cryoablation device. In those embodiments in which the system 10 is typically maintained in a storage area, at this point, the system 10 is transported into the procedure room and reconnected to electrical power and the cryoablation device 16 is coupled to the container unit 14. More specifically, the connector section 110 is coupled to the interface 30 such that the lines 112-120 are coupled to the ports 68-76 as discussed in detail above.

Once the ablation device 16 is coupled to the unit 14, in certain embodiments, a vacuum can be applied to the interior of the interface 30. When the ablation procedure begins, the gas pressure accumulation within the container 400 causes the cryogenic liquid to pass from the bottom of the inner chamber 404 into lance 402 In this embodiment, when combining the container 400 of FIG. 4 into the system of FIG. 1, lance 402 is connected to cold connection line 60, via interface 30. When ablation device 16 is connected via connection unit 110, cold line 120 now becomes the cold inflow line. As the cryogenic liquid is urged through the system, it boils and converts to very cold gas and passes through ablation device 16 and is exhausted via cold line 114, which connects via interface 30 to return the cold exhaust to unit 14 for collection and handling, but not necessarily into a recovery container. At this point, according to some embodiments, a limited volume of cryogenic gas returning from the ablation device 16 may be allowed to pass via the valve 64 and first warm connection line 48 into the accumulator 24 for later use, as described above.

According to certain embodiments, between each cold cycle during an ablation procedure, a warm/thaw cycle can also be provided as described in further detail above such that the ablation device (such as device 16) can be moved and re-positioned in order to find the next tissue ablation target. Once it is positioned as desired, another cold cycle can be initiated. Once the ablation procedure is complete, in certain implementations, the system 10 may be moved back into its storage location, where it can be filled with an external source of helium gas to be converted to liquid for future use, and where it is re-connected to an electrical supply and placed in a storage/recuperation mode as discussed in detail above.

In accordance with yet another alternative implementation, the system 10 as described in detail above with respect to FIGS. 1A and 1B can have another configuration. That is, instead of having a recovery/liquefication container (such as container 18) and a working supply container (such as container 20), the system 10 can have two containers similar to the recovery/liquefication container, with both having cold heads (similar to cold head 98 as described above). Thus, both containers can be incorporated into the system 10 in a fashion similar to the two containers 18, 20 as described above, except that each can cool the cryogenic fluid to different temperatures, according to different types of embedded cold head units (i.e., one being 4 K and the other being 26 K, for example).

The various system embodiments disclosed or contemplated herein can utilize XLT cryofluid for cryoablation procedures while overcoming limitations that heretofore have precluded the safe use of such XLT cryofluids in a hospital setting. Variations of these systems are contemplated, including systems that take advantage of the described innovations regarding cryogenic liquid generation and transfer and warm cryogenic fluid conservation while forgoing the challenging aspects of cold exhaust recuperation and preventing cold cycle cryofluid from being exhausted to the environment.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

Although the various embodiments have been described with reference to preferred implementations, persons skilled in the art will recognize that changes may be made in form and detail without departing from the spirit and scope thereof.

1. A cryogenic fluid cooling system for ablation procedures, the system comprising a mobile unit comprising:
a) a first cryogenic cooling container comprising:
i) a first inner chamber;
ii) a cryogenic fluid cooling unit disposed within the first inner chamber;
iii) a first elongate outlet tube extending into the first inner chamber, the first elongate outlet tube comprising an outlet opening disposed at or near the bottom of the first inner chamber; and
iv) a first inlet tube extending into the first inner chamber, the first inlet tube comprising an inlet opening disposed at or near the cryogenic fluid cooling unit; and
b) an interface associated with the mobile unit and detachably coupleable to an ablation device, the interface comprising:
i) an interface interior; and
ii) a transfer port in fluidic communication with the first inner chamber via the first elongate outlet tube, wherein the transfer port is removably coupleable with the ablation device,
wherein the first cooling container is configured to cool the cryogenic fluid disposed in the first inner chamber and retain the cryogenic fluid in a liquid or solid state for any period of time, and
wherein the system is configured to urge cryogenic fluid in the liquid state via vapor pressure accumulation from the first inner chamber into the ablation device via the transfer port when the ablation device is coupled to the interface.

2. The cryogenic fluid cooling system of claim 1, wherein the vapor pressure accumulation occurs above a surface of the cryogenic fluid in the liquid state in the first inner chamber.

3. The cryogenic fluid cooling system of claim 1, wherein the cryogenic fluid is exhausted in a gas state from the ablation device back into the cooling system.

4. The cryogenic fluid cooling system of claim 1, wherein the cryogenic fluid is helium, neon, or hydrogen.

5. The cryogenic fluid cooling system of claim 1, wherein first inner chamber comprises an inner wall configured to withstand internal pressures of up to around 100 psi.

6. The cryogenic fluid cooling system of claim 1, further comprising a collection unit comprising:
a) a second cooling container comprising:
A) a second inner chamber; and
B) a second cryogenic fluid cooling unit disposed within the second inner chamber; and
b) an intake interface comprising at least one intake port coupleable with an exhaust cryogenic fluid source, wherein the at least one intake port is in fluidic communication with the second inner chamber; and
c) a compressor unit operably coupled to the second cryogenic fluid cooling unit.

7. The cryogenic fluid cooling system of claim 6, wherein the exhaust cryogenic fluid source is the ablation device or a container containing the exhaust cryogenic fluid from the ablation device.

8. The cryogenic fluid cooling system of claim 1, further comprising a controller associated with the mobile unit, wherein the controller is configured to control flow of the cryogenic fluid within the system.

9. The cryogenic fluid cooling system of claim 1, wherein the cryogenic fluid cooling unit comprises a cryocooler cold head unit.

10. The cryogenic fluid cooling system of claim 1, wherein the system is configured to minimize or eliminate external loss of the cryogenic fluid.

11. The cryogenic fluid cooling system of claim 1, wherein the system is configured to receive the spent cryogenic fluid from the ablation device and transfer the spent cryogenic fluid into at least one exhaust fluid container configured to receive the spent cryogenic fluid via a fluid container exhaust interface.

12. A cryogenic fluid cooling system for ablation procedures, the system comprising a mobile unit comprising:
a) a cryogenic storage container comprising:
i) an inner chamber configured to be refillable with cryogenic fluid;
ii) an elongate tube extending into the inner chamber, the elongate tube comprising an opening disposed at or near the bottom of the inner chamber;
b) an interface associated with the mobile unit and detachably couplable to an ablation device, the interface comprising:
i)
a transfer port in fluidic communication with the inner chamber via the elongate tube, wherein the transfer port is removably coupleable with the ablation device; and
ii) a return port removably coupleable to the ablation device, wherein the return port is configured to collect spent cryogenic fluid,
wherein the cryogenic storage container is configured to retain the cryogenic fluid in a liquid state for any period of time, and
wherein the system is configured to urge the cryogenic fluid in the liquid state via pressure accumulation in the inner chamber into the ablation device via the transfer port when the ablation device is coupled to the interface.

13. The cryogenic fluid cooling system of claim 12, wherein the pressure accumulation occurs due to a volume of gas added to the inner chamber and occupying space above a surface of the cryogenic fluid in the liquid state stored in the inner chamber.

14. The cryogenic fluid cooling system of claim 12, wherein the cryogenic fluid is exhausted in a gas state from the ablation device back into the cooling system via the return port.

15. The cryogenic fluid cooling system of claim 12, wherein the cryogenic fluid is helium, neon, or hydrogen.

16. The cryogenic fluid cooling system of claim 12, wherein inner chamber comprises an inner wall configured to withstand internal pressures of up to around 100 psi.

17. A method of cooling, storing, supplying and recovering cryogenic fluid, the method comprising:
cooling cryogenic gas with a cold head unit in a cooling container of a cooling system to a temperature of less than 27 Kelvin such that the cryogenic gas is converted into cryogenic liquid or solid;
retaining the cryogenic liquid or solid in the cooling container for any period of time;
transferring the cryogenic liquid into a supply container prior to an ablation procedure;
transferring the cryogenic liquid into an ablation device via vapor pressure accumulation in the supply container, wherein the cryogenic liquid is converted into cryogenic gas as it passes through the ablation device; and
receiving the cryogenic gas in the cooling system from the ablation device,
wherein any external loss of the cryogenic fluid is minimized or prevented.

18. The method of claim 17, wherein the cryogenic fluid is helium, neon, or hydrogen.

19. The method of claim 17, further comprising immediately cooling the cryogenic gas received in the cooling system from the ablation device.

20. The method of claim 17, further comprising cooling the cryogenic gas received in the cooling system from the ablation device in a cryocooler for future reuse.
